(19) United States
(12) Patent Application Publication
(10) Pub. No.: US 2025/0082734 A1
(43) Pub. Date: Mar. 13, 2025

(54) PTPRS IN AUTOIMMUNITY

(71) Applicant: The Regents of the University of California, Oakland, CA (US)

(72) Inventor: Nunzio Bottini, La Jolla, CA (US)

(21) Appl. No.: 18/290,679

(22) PCT Filed: Jul. 29, 2022
(86) PCT No.: PCT/US2022/074351

§ 371 Date: Jan. 19, 2024

Related U.S. Application Data
(60) Provisional application No. 63/227,532, filed on Jul. 30, 2021.

(51) Int. Cl. A61K 38/46; A61K 38/17; C07K 14/705; C12N 9/16

(52) U.S. Cl. CPC A61K 38/465; A61K 38/177; C07K 14/705; C12N 9/16; C12Y 301/03048

(57) ABSTRACT

The methods and compositions of the disclosure are used to prevent or treat autoimmune diseases and disorders, such as lupus, by administering a PTPRS activating agent. The agent does not deplete pDCs or cause a general blockade of IFNα signaling and therefore is less immunosuppressive and easier to combine with other immunosuppressants.

### CROSS REFERENCE TO RELATED APPLICATIONS

This application claims priority to U.S. Provisional Application Ser. No. 63/227,532, filed Jul. 30, 2021, the disclosures of which are incorporated herein by reference.

### TECHNICAL FIELD

Provided herein are compositions and methods useful for modulating and treating autoimmune diseases and disorders and more particularly for treating TLR9-associated disease and disorders such as lupus.

### INCORPORATION BY REFERENCE OF SEQUENCE LISTING

Accompanying this filing is a Sequence Listing entitled, “00015-404WO1.xml” created on Jan. 29, 2022 and having 15,747 bytes of data, machine formatted on IBM-PC, MS-Windows operating system using WIPO Standard ST.26 formatting. The sequence listing is hereby incorporated by reference in its entirety for all purposes.

### BACKGROUND

Approximately 5 to 8% of people in the United States suffer from an autoimmune disease. Researchers have identified more than 80 different autoimmune diseases and suspect that many more diseases may have an autoimmune component. Lupus is an autoimmune disease that affects over 1.5 million Americans and at least 5 million people worldwide. Women are especially at higher risk, with individuals in their childbearing years being most affected. Approximately 90% of all individuals affected by lupus are women.

Lupus is an autoimmune disease, specifically an inflammatory disease caused when the immune system attacks its own tissue. Systemic Lupus Erythematosus (SLE) can affect the joints, skin, kidneys, blood cells, brain, heart, and lungs.

Symptoms vary but can include fatigue, joint pain, rash, and fever. These can periodically get worse (flare-up) and then improve.

### SUMMARY

The disclosure provides compositions, methods and kits comprising PTPRS activating agents. The agents of the disclosure are recombinant proteins comprising an amino acid sequence of an extracellular domain of PTPRS or a portion thereof.

The disclosure provides a method for activating PTPRS on immune cells called plasmacytoid dendritic cells (pDCs) which is relevant for the pathogenesis of lupus and other autoimmune diseases. pDCs are high producers of cytokines involved in the pathogenic autoimmune phenotypes, including interferon alphas (IFNα; also referred to as Type I), interleukin 6 (IL-6), tumor necrosis factor α (TNF), and interferon gamma (IFN-γ). Patients with autoimmune disorders often present with an “interferon positive signature” (IFN signature) which is detected in the blood using lab tests. Depletion of pDCs or blockages of IFNα signaling are in advanced trials for lupus and other IFN signature positive patients. However, systemic depletion of pDCs and systemic blockage of IFNα generally make a subject susceptible to infections and other disease and disorders. Accordingly, selective targeting of pathogenic cytokines, including IFNα produced by pDCs, would improve therapy.

PTPRS is known to be expressed at high levels in pDCs and its knockout increases release of IFNα from these cells.

PTPRS is regulated by binding to proteoglycans on the surfaces of fibroblasts and neurons, although prior to this disclosure nothing was known about proteoglycans and PTPRS in pDCs. The disclosure demonstrates that dissociation of PTPRS from proteoglycans using a decoy agent is sufficient to inhibit stimulation-induced release of IFNα from pDCs and protect, in an IFNα- and pDC-dependent model of lupus, from development of severe disease.

The methods and compositions of the disclosure can be used to reduce severity of lupus and other autoimmune diseases. Some patients with lupus or other autoimmune diseases are identified by the IFN signature, but it is understood that the disclosed methods and compositions can treat all patients diagnosed with lupus or other autoimmune diseases, regardless of whether they exhibit the IFN signature.

The disclosure provides a method of treating an autoimmune disease in a subject, the method comprising administering to the subject a therapeutically effective amount of a PTPRS activating agent, wherein administration treats the autoimmune disease in the subject. In one embodiment, the autoimmune disease is lupus. In another embodiment, the autoimmune disease is systemic lupus erythematosus. In still another embodiment, the autoimmune disease has an IFN signature. In yet another embodiment, the subject has a TLR9-associated disease or disorder that causes increased IFNα production. In another embodiment, the PTPRS activating agent inhibits the association of proteoglycans with PTPRS on pDCs. In another embodiment, the PTPRS activating agent comprises a soluble extracellular domain of PTPRS. In a further embodiment, the agent is selected from the group consisting of PTPRS Ig1&2, PTPRS Ig1&2&3, PTPRS Ig2&3, and PTPRS Ig1&3 and multimers thereof. In still a further embodiment, the agent is PTPRS Ig1&2. In yet a further embodiment, the agent comprises multimers of Ig1&2 wherein the multimers are linked by a peptide linker.

The disclosure also provides a composition comprising a PTPRS activating agent, e.g., for use in the method of the disclosure. In one embodiment, the PTPRS activating agent inhibits the association of proteoglycans with PTPRS on pDCs. In another embodiment, the PTPRS activating agent comprises a soluble extracellular domain of PTPRS. In still another embodiment, the agent is selected from the group consisting of PTPRS Ig1&2, PTPRS Ig1&2&3, PTPRS Ig2&3, and PTPRS Ig1&3 and multimers thereof. In a further embodiment, the agent is PTPRS Ig1&2. In yet a further embodiment, the agent comprises multimers of Ig1&2 wherein the multimers are linked by a peptide linker.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1 provides a graph in which PTPRS Ig1&2 inhibits IFNα induction by CpGA in mouse pDCs. After sorting from Balb/c spleens, pDCs were treated with or without 5 μM CpGA±20 nM Ptprs Ig1&2. IFNα expression relative to GAPDH was assessed by qPCR. Mean±SEM is shown. * p<0.05, Mann-Whitney test.

FIG. 2 provides experimental results where PTPRS Ig1&2 inhibits CpGA-induced IFNα release by mouse pDCs in vivo. Samples were collected from Balb/c mice after treatment with or without 20 nmols CpGA±500 μg PTPRS Ig1&2. IFNα release in sera was measured by ELISA. Mean±SD is shown. * p<0.05, unpaired t-test. Flow analysis confirmed no effect of Ig1&2 on pDC viability in isolated splenocytes as assessed by Live/Dead staining.

FIG. 3A shows proteinuria results for lupus-prone BXSB mice after Ig1&2 treatment vs control (n=3 per group). Assessment of proteinuria in the same mice prior to treatment confirmed comparable levels between the two groups. * p<0.05, unpaired t-test.

FIG. 3B shows spleen weight in Ig1&2 treated and control mice (n=3 per group).

FIG. 4A provides representative kidney histology images following Periodic acid-Schiff (PAS) staining to detect immune cell infiltration in kidneys of control and Ig1&2 treated BXSB mice.

FIG. 4B shows quantification of kidney infiltration by histological score of kidneys of control and Ig1&2 treated BXSB mice (n=3 per group). * p<0.05, unpaired t-test.

FIG. 5 provides a graph showing that PTPRS Ig1&2 inhibits IFNα inducation by CpGA in human pDCs. PBMC were purified with ficoll/histopaque and pDCs were isolated by negative selection. Cells were activated with or w/o 5 microM CpGA±20 nM PTPRS Ig1&2. IFNα gene expression relative to GAPDH was measured by qPCR. Mean±SEM is shown. ** p<0.01, Mann-Whitney test. pDC viability percentage was confirmed by Trypan Blue Assay on a Vi-CELL XR 2.03.

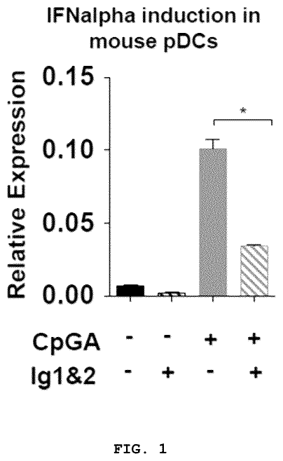

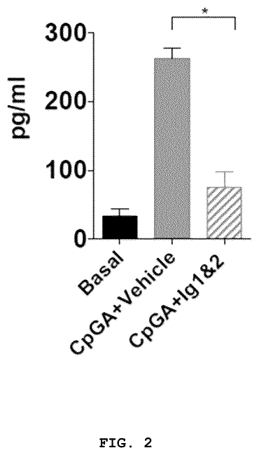

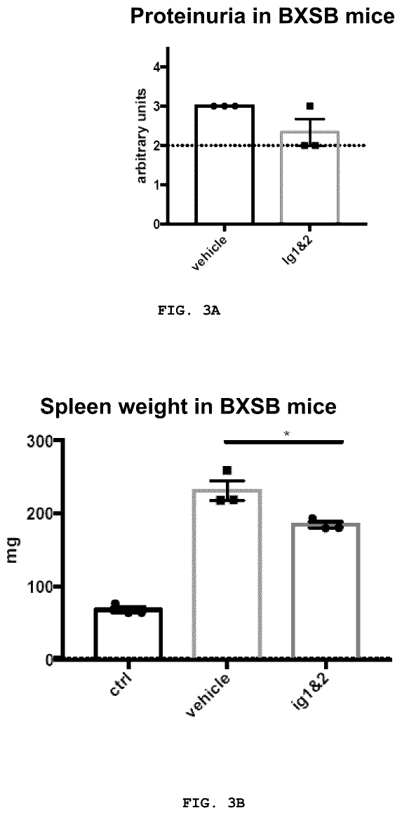

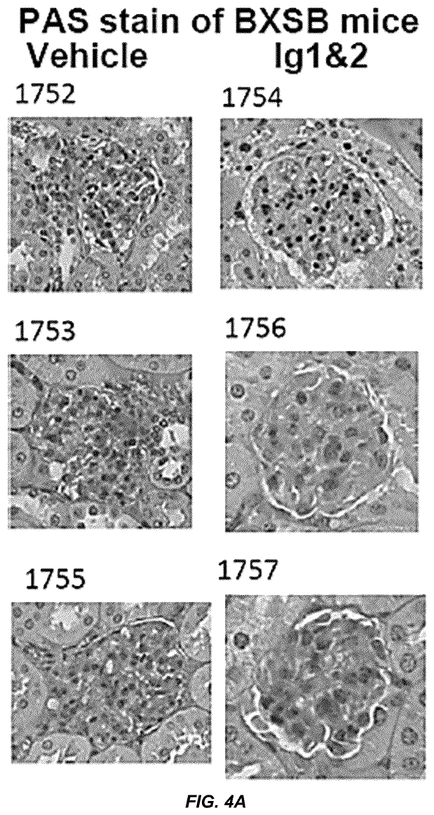

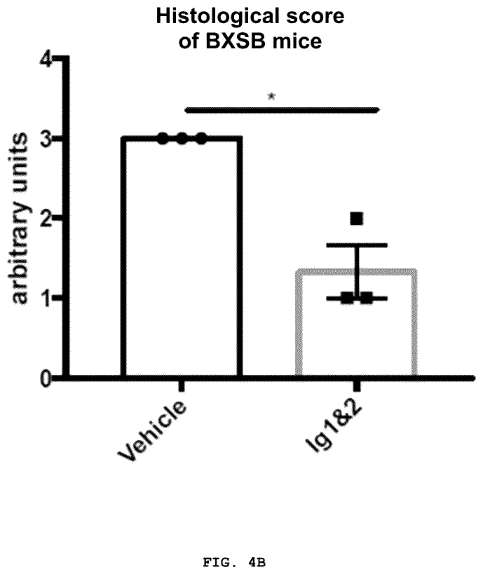

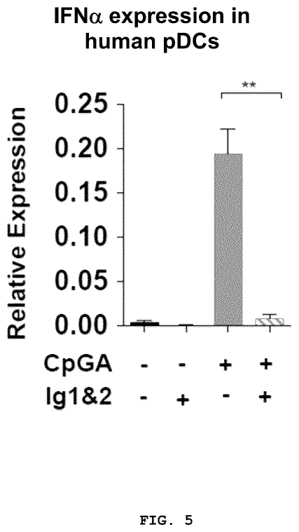

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the agent” includes reference to one or more agents, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

Regulation of PTPRS by the proteoglycan switch in pDCs is as follows: PTPRS is specifically expressed in pDCs where it inhibits TLR9-induced type I IFN release. The activity of PTPRS (i.e., inhibiting TLR9-induced type I IFN) is physiologically inhibited by its binding to surface proteoglycans. Treatment of pDCs with a decoy or PTPRS activating agent as described herein, such as Ig1&2, dissociates PTPRS from proteoglycans and activates the phosphatase, resulting in inhibition of TLR9-induced type I IFN release.

Provided herein are methods of modulating PTPRS activity in a subject, the method comprising administering to the subject an effective amount of a PTPRS activating agent, wherein administration modulates PTPRS activity in the subject. Also provided are methods of treating, preventing, and/or ameliorating an autoimmune disease or disorder in a subject in need thereof. Provided is a method of preventing or treating an autoimmune disease in a subject, the method comprising administering to the subject a therapeutically effective amount of a PTPRS activating agent, wherein administration treats the autoimmune disease in the subject. Also provided is a method of treating an autoimmune disease in a subject, the method comprising administering to the subject a therapeutically effective amount of a compound that increases PTPRS phosphatase activity, wherein administration treats the autoimmune disease in the subject. Autoimmune diseases or disorders include, but are not limited to, inflammatory autoimmune diseases. The autoimmune disease can be selected from arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma. Optionally, the autoimmune disease is arthritis, Crohn's disease, scleroderma, or rheumatoid arthritis.

The methods include administering an effective amount of the provided agents and compositions, wherein administering the effective amount of the composition treats or prevents the autoimmune disease in the subject. Administration of a composition disclosed herein can be a systemic or localized administration. For example, treating a subject having an inflammatory autoimmune disorder can include administering an oral or injectable form of the pharmaceutical composition on a daily basis or otherwise regular schedule. In some embodiments, the treatment is only on an as-needed basis, e.g., upon appearance of inflammatory autoimmune disease symptoms.

Also provided are methods of decreasing IFNα production in a subject. The methods include administering to the subject a therapeutically effective amount of a PTPRS activating agent, wherein administration decreases IFNα production in the subject.

In some cases, the subject has a TLR9-associated IFNα over-production disease. Thus, provided are methods of treating a TLR9-associated disease in a subject. The methods include administering to the subject a therapeutically effective amount of a PTPRS activating agent, wherein administration treats the TLR9-associated disease in the subject.

As used herein the term “agent” refers to any molecule or compound that can be used in the methods and compositions of the disclosure to inhibit the binding of proteoglycans to PTPRσ on pDC cells. An agent can be a biological agent such as a protein, peptide, polypeptide (e.g., an antibody or fragment thereof), nucleic acid (e.g., RNAi molecule); a macromolecule or small molecule agent. An agent can be referred to as a decoy, e.g. “a decoy agent”. In an embodiment, the agent comprises the soluble domain (SD) of PTPRσ. In embodiments, the “agent” comprises a soluble peptide or polypeptide of PTPRσ. In embodiments, the soluble PTPRS peptide or polypeptide of the disclosure may comprise one, two, or three immunoglobulin-like domains in various combinations. In some embodiments, the soluble PTPRS polypeptide comprises the first (Ig1), second (Ig2), and third (Ig3) Ig-like domains of PTPRS (referred to as “Ig1&2&3”). In an embodiment, the soluble PTPRS polypeptide Ig1&2&3 comprises a polypeptide of SEQ ID NO:13. In other embodiments, the soluble PTPRS polypeptide Ig1&2&3 can be modified to reduce susceptibility to proteases. In an embodiment, the soluble PTPRS polypeptide Ig1&2&3 comprises SEQ ID NO: 14. In other embodiments, the soluble PTPRS peptide or polypeptide may comprise Ig1 and Ig2 (referred to as “Ig1&2”). In certain embodiments, the soluble PTPRS peptide or polypeptide may comprise the Ig1 and Ig3 (referred to as “Ig1&3”). In other embodiments, the soluble PTPRS peptide or polypeptide may comprise Ig2 and Ig3 (referred to as “Ig2&3”). In still other embodiments, the soluble PTPRS peptide or polypeptide of the disclosure may comprise each Ig-like domain alone (e.g., Ig1 or Ig2 or Ig3).

In certain embodiments, the order of the 3 PTPRS Ig-like domains is interchangeable. For example, the term “Ig1&2” may refer to either or both of two constructs; i.e. a construct in which the amino acid sequences can be arranged in one of two configurations, that is, with Ig1 at the N-terminus and Ig2 at the C-terminus, or with Ig2 at the N-terminus and Ig1 at the C-terminus. Similarly, the term “Ig1&2&3” may refer to any one or more of six constructs, in which the three extracellular Ig-like domains are ordered, from N-terminus to C-terminus, in any one of the following configurations: Ig1&2&3, Ig1&3&2, Ig2&3&1, Ig2&1&3, Ig3&1&2, and Ig3&2&1. A PTPRS activating agent can comprise one or more of these constructs.

The soluble PTPRS Ig-like domain polypeptides or peptides may be present or be produced as oligomers, such as dimers and trimers. The oligomers may be formed by noncovalent interactions under conditions that favor such interactions (which include physiological conditions) or may form by a combination of covalent and non-covalent interactions. Alternatively, oligomers may be formed by chemically or recombinantly linking at least two monomeric RPTPS Ig-like domain polypeptides. The oligomers may comprise, for example, homodimers or heterodimers. For instance, a homodimer may comprise (1) a first monomer selected from Ig1, Ig2, and Ig3 of RPTPS and (2) a second monomer that is the same as the first monomer. A heterodimer comprises (1) a first monomer selected from Ig1, Ig2, and Ig3 of PTPRS and (2) a second monomer selected from Ig1, Ig2, and Ig3 of PTPRS, but wherein the monomer selected in (1) and in (2) are not the same. The soluble PTPRσ Ig-like domain-containing peptides and polypeptides of this disclosure may be prepared recombinantly using molecular biology techniques or may be noncovalently combined or covalently fused with or without one or more linking or spacer amino acids between one or more of the domains.

As used herein “autoimmune disease” refers to a type of disease that occurs as a result of an immune response by the body against its own tissue, cell, or cell components. Normally, the body's immune cells carry out a set of immune responses against pathogenic microbes or antigens that gain entry into the body. However, in the case of autoimmune diseases, the immune cells are activated against the host's body rather than a foreign pathogen. These responses include, but are not limited to, antibody production, induction of cell-mediated immunity, and/or complement activation.

As used herein “cytokine” generally refers to cell signaling molecules that aid cell to cell communication in immune responses and stimulate the movement of cells towards sites of inflammation, infection and/or trauma.

As used herein the term “diagnosis” refers to a relative probability that a disease or disorder (e.g. an autoimmune, inflammatory autoimmune, cancer, infectious, immune, or other disease) is present in a subject. The disclosure provides methods of diagnosing a disease or disorder associated with IFNα over-production, such as lupus. Diseases with IFNα over-production include, but are not limited to, diseases associated with the IFN signature, scleroderma, inflammatory myositis, and Sjogren syndrome. Diseases with IFNα over-production can be treated using the methods and compositions of the disclosure to inhibit binding of proteoglycans to PTPRσ. While not wishing to be held to a specific theory, such inhibition of binding between PTPRσ and proteoglycans, for example those found in the extracellular matrix, induces declustering of PTPRσ, thereby activating PTPRσ and leading to signaling inhibition, including the reduction of release of IFNα and/or other cytokines.

As used herein the terms “dose” and “dosage” are used interchangeably. A dose refers to the amount of active ingredient (e.g., PTPRσ Ig1&2) given to an individual at each administration, or to an amount administered in vitro or ex vivo. For the methods and compositions provided herein, the dose may generally depend on the required treatment for the disease (e.g. an autoimmune, inflammatory autoimmune, or other disease), and the biological activity of a compound or agent disclosed herein. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress.

As used herein the term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “effective amount,” “therapeutically effective amount,” “therapeutically effective dose or amount” and the like as used herein is meant as an amount (e.g., a dose) that produces effects for which it is administered (e.g., inhibiting interaction of proteoglycans on pDCs with PTPRσ). An effective dose can be characterized in cell culture to modulate a particular biological readout (e.g., enzymatic activity, clustering, signaling, IFNα production, and other pathogenic cytokines, for example IL-6, TNF, IFN-gamma). The exact dose and formulation will depend on the purpose of the research or treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 58, 108, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

The term “interferon alpha (IFNα)” refers to a family of cytokines which inhibit viral replication, suppress cell proliferation, and regulate the immune response to various diseases.

As used herein “lupus” is a chronic autoimmune disease that can affect many organs, including the skin, joints, central nervous system, heart, lungs, gastro-intestinal symptoms, and kidneys. Lupus is a clinically heterogenous disease with diverse features, including inflammation, changes in complement levels, the presence of autoantibodies, cutaneous manifestations, and others. Lupus can occur as distinct disease subsets, including cutaneous lupus and drug-induced lupus.

As used herein “pathogenesis” is the process by which a disease or disorder develops. It can include factors which contribute not only to the onset of the disease or disorder, but also to its progression and maintenance.

As used herein “plasmacytoid dendritic cell (pDC)” is a rare type of immune cell known to secrete large quantities of type 1 interferon (IFNα) in response to a viral infection. pDCs circulate in the blood and are found in peripheral lymphoid organs.

As used herein “proteoglycans” are proteins that are heavily glycosylated. The basic proteoglycan unit consists of a “core protein” with one or more covalently attached glycosaminoglycan chain. The point of attachment is a serine residue to which the glycosaminoglycan is joined through a tetrasaccharide bridge. Proteoglycans important in the proteoglycan switch mechanism for regulation by PTPRσ are heparan sulfate (HS), which is layered on the cell surfaces of many cell types, and chondroitin sulfate (CS), which is in the extracellular matrix of certain cell types.

As used herein “PTPRS” also known as: Receptor-type tyrosine-protein phosphatase S, R-PTP—S, R—PTP-sigma, PTPRσ, or RPTPσ, is an enzyme that in humans is encoded by the PTPRS gene. The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. PTPRσ contains an extracellular region, a single transmembrane segment and two tandem intracytoplasmic catalytic domains (D1 and D2), and thus represents a receptor-type PTP. D1 is catalytically active, while D2 is catalytically inactive. The extracellular region of this protein is composed of multiple Ig-like and fibronectin type III-like domains.

The amino acid sequence of PTPRS can be found, for example, at UniProtKB/Swiss-Prot Accession No. Q13332 and BOV2N1. The nucleic acid sequence of PTPRS can be found, for example, at GenBank Accession No. NC 000019.9 PTPRS includes an intracellular domain, a transmembrane domain and an extracellular domain. The term transmembrane domain refers to the portion of a protein or polypeptide that is embedded in and, optionally, spans a membrane. The term intracellular domain refers to the portion of a protein or polypeptide that extends into the cytoplasm of a cell. The term extracellular domain refers to the portion of a protein or polypeptide that extends into the extracellular environment. The extracellular domain of PTPRS includes immunoglobulin-like domain 1 (Ig1), immunoglobulin-like domain 2 (Ig2) and immunoglobulin-like domain 2 (Ig3). In an embodiment, the amino acid sequence of Ig1 is SEQ ID NO:1. In an embodiment, the amino acid sequence of Ig2 is SEQ ID NO: 2. In an embodiment, the amino acid sequence of Ig3 is SEQ ID NO: 3. Isoforms of the Ig-like domains of PTPRS are known in the art (e.g. Palido R, et al. 1995 Proc. Natl. Acad. Sci. 92:11686-90). Such isoforms can be used interchangeably in the constructs of PTPRS activating agents.

As used herein, the term “non-enzymatic recombinant protein” refers to a recombinant protein that does not have enzymatic activity (e.g., the protein does not function as a biological catalyst). Thus, in some embodiments, the non-enzymatic recombinant proteins comprising an amino acid sequence of an extracellular domain of PTPRS include only extracellular domain portions of the PTPRS and not the enzymatic portions of the PTPRS. In embodiments, the non-enzymatic recombinant proteins comprising an amino acid sequence of an extracellular domain of PTPRS include only extracellular domain portions of the PTPRS and not the enzymatic portions of the PTPRS or the transmembrane portions of the PTPRS. In some embodiments, the non-enzymatic recombinant proteins comprising an amino acid sequence of an extracellular domain of PTPRS include two or more extracellular domain of PTPRS linked together (e.g. linked by an amino acid linker such as an amino acid linker of at least 2, at least 3, at least 5, at least 10, about 2 to 20 or 2 to 30, or 2 to 50 amino acids, about 3 to 50 amino acids, or about 2, 3, 4 5, 6, 7, 8, 9, 10, 20, 30, 40, or 50 amino acids wherein the amino acid sequence is designed to not interfere with extracellular domain of PTPRS ligand binding and the linker sequence itself is preferably not immunogenic.

The term “extracellular domain of PTPRS” can include subsequences, portions, homologues, isoforms, variants or derivatives of the extracellular domain of PTPRS. Thus, the non-enzymatic recombinant protein can comprise a portion of the extracellular domain of PTPRS, e.g., the protein comprises one or more immunoglobulin-like domains of PTPRS, or subsequences, portions, homologues, isoforms, variants or derivatives thereof. The extracellular domain of PTPRS is typically capable of binding (e.g., specifically binding) to a PTPRS ligand such as a proteoglycan. Optionally, the extracellular domain of PTPRS comprises one or more of PTPRS immunoglobulin-like domain 1 (Ig1), immunoglobulin-like domain 2 (Ig2) and immunoglobulin-like domain 2 (Ig3), or a subsequence, portion, homologue, variant or derivative thereof. Optionally, the extracellular domain of PTPRS comprises one or both of PTPRS immunoglobulin-like domain 1 (Ig1) and immunoglobulin-like domain 2 (Ig2) or a subsequence, portion, homologue, variant or derivative thereof.

As used herein, “PTPRσ Ig1&2”, “PTPRσ Ig1&3”, “PTPRσ Ig2&3” and “PTPRσ Ig1&2&3” refer to a soluble domain of PTPRσ (e.g., lacking the intracellular and transmembrane portions of PTPRσ). It is understood that many mammalian species have a PTPRS gene and produce PTPRS proteins with three extracellular Ig-like domains. In various embodiments, PTPRσ Ig1&2 comprises SEQ ID NO: 4 (human), SEQ ID NO: 5 (mouse), SEQ ID NO: 6 (chimpanzee), SEQ ID NO: 7 (monkey), SEQ ID NO: 8 (dog), SEQ ID NO:9 (cow), SEQ ID NO:10 (rat), SEQ ID NO:11 (chicken), or SEQ ID NO:12 (horse). The disclosure provides a plurality of constructs comprising PTPRσ Ig1&2, PTPRσ Ig1&3, PTPRσ Ig2&3, and PTPRσ Ig1&2&3 having various modified linkers between the specified Ig-like domains of PTPRσ.

As used herein a “PTPRS activating agent” refers to an agent that inhibits binding of proteoglycans to PTPRS (e.g., inhibits PTPRS-proteoglycan association), thereby allowing PTPRS to be active and inhibit intracellular signaling. In one embodiment, the proteoglycan and PTPRS are present on pDCs. In another embodiment, the PTPRS activating agent comprises a soluble extracellular domain of PTPRσ, as described herein.

The terms “subject,” “patient,” “individual,” etc. are not intended to be limiting and can be generally interchanged. That is, an individual described as a “patient” does not necessarily have a given disease, but may be merely seeking medical advice or may have a predisposition to a given disease. Moreover, a subject, patient or individual can be any mammal including primates, canines, felines, bovines, equines, porcine, etc. Preferably the subject is a human subject.

TLR9 (also sometimes referred to in the literature as CD289) is a receptor expressed in immune system cells including pDCs and B cells. B cells, also referred to as B lymphocytes, are a type of white blood cell of the lymphocyte subtype. They generally function in the adaptive immune system (i.e., humoral immunity component) by secreting antibodies. B cells can present antigen and secrete cytokines. In mammals, B cells mature in the bone marrow. B cells typically express B cell receptors (BCRs) on their cell membrane. BCRs allow the B cell to bind to a specific antigen and initiate an antibody response.

The term “TLR9,” as used herein, refers to any native, mature TLR9 that results from processing of a TLR9 precursor protein in a cell. The term includes TLR9 from any vertebrate source, including mammals such as primates (e.g., humans and cynomolgus or rhesus monkeys) and rodents (e.g., mice and rats), unless otherwise indicated. The term also includes naturally occurring variants of TLR9, e.g., splice variants or allelic variants.

The term “TLR9-positive cell” refers to any cell that expresses TLR9 on its surface or on an intracellular membrane or organelle (e.g., endosome, ER, Golgi apparatus, lysosome, and the like). Some cells, including those infected by a microbe or associated with some cancer types and tumors, exhibit up-regulation of TLR9 expression.

As used herein, the term “TLR9-associated diseases” refers diseases associated with over active TLR9 biological activity. In some embodiments, the excess biological activity of TLR9 increases the amount of IFNα secreted by a cell. Examples of TLR9-associated diseases include, but are not limited to, autoimmune diseases, autoimmune inflammation, autoimmune thyroid diseases. Specific example of diseases and disorders that can be treated by the methods and compositions of the disclosure include, but are not limited to, rheumatoid arthritis, spondyloarthropathies, systemic lupus erythematosus, allergic asthma, allergic rhinitis, atopic dermatitis, ulcerative colitis, vascular restenosis, type I diabetes, type II diabetes, urticaria, conjunctivitis, psoriasis, inflammatory myositis, dermatomyositis, mixed connective tissue disease, Sjogren's syndrome, gout, diabetic retinopathy, multiple sclerosis, Crohn's disease, chronic thyroiditis, celiac disease, myasthenia gravis, pemphigus vulgaris, viral disease, bacterial disease, radiation damage, and cardiac hypertrophy.

As used herein, the terms “treat” and “prevent” may refer to any delay in onset, reduction in the frequency or severity of symptoms, amelioration of symptoms, improvement in patient comfort or function (e.g., joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g., an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

This disclosure describes the surprising finding that proteoglycans on pDCs bind to and prevent the biological activity of PTPRS on pDCs. The sequestering, or clustering, of PTPRS by proteoglycans inhibits the downregulation of TLR9 by PTPRS and thus leads to production of IFNα. PTPRS mediates signaling on pDC through its enzymatic activity on TLR9 signaling components. When PTPRS is clustered on pDC (through binding with proteoglycans), its phosphatase activity is inhibited and TLR9 signaling within the pDC cell proceeds, leading to the production of IFNα. When PTPRS is de-clustered on pDC, through the agents of this disclosure, PTPRS is enzymatically active thereby inhibiting TLR9 signaling, leading to a reduction in the production of IFNα.

The disclosure provides a method of treating an autoimmune disease in a subject, the method comprising administering to the subject a therapeutically effective amount of an agent that inhibits the association of proteoglycans and PTPRS. In embodiments, the method of the disclosure comprises administering a therapeutically effective amount of a PTPRS-activating agent. The disclosure provides a method of treating an autoimmune disease selected from the group consisting of lupus, systemic lupus erythematosus, and diseases with an IFN signature in a subject, the method comprising administering to the subject a therapeutically effective amount of a PTPRS activating agent. In certain embodiments, the method of the disclosure comprises administering a therapeutically effective amount of a soluble extracellular domain of PTPRS. In other embodiments, the method of the disclosure comprises administering a therapeutically effective amount of a PTPRS activating agent selected from the group consisting of PTPRS Ig1&2, PTPRS Ig1&2&3, PTPRS Ig2&3, and PTPRS Ig1&3 and multimers thereof. In yet other embodiments, the disclosure provides a method of treating an autoimmune disease in a subject, the method comprising administering to the subject a combination of (1) one or more doses of an effective amount of a composition comprising a PTPRS activating agent and (2) an immunosuppressant. In still other embodiments, the disclosure provides a method of treating an autoimmune disease in a subject, the method comprising administering to the subject a combination of (1) one or more doses of an effective amount of a composition comprising a PTPRS activating agent and (2) anti-B cell agents, for example a TNF inhibitor or IL-6 inhibitor. While not wishing to be held to a particular theory, these combinations are preferred because the PTPRS activating agent does not deplete pDCs or cause a general blockade of IFNα signaling and therefore is less immunosuppressive and easier to combine with other immunosuppressants.

The disclosure provides compositions including the agents provided herein for use in treating diseases. Provided herein are pharmaceutical compositions including a PTPRS-activating agent and a pharmaceutically acceptable excipient. The compositions can include additional agents. The compositions are, optionally, suitable for formulation and administration in vitro or in vivo. Optionally, the compositions comprise one or more of the provided agents and a pharmaceutically acceptable carrier. Suitable carriers and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

In certain embodiments, the PTPRS activating agents are administered in a combination amount with other agents, i.e., a secondary agent, useful for treating the targeted diseases and disorders. The PTPRS activating agent and the secondary active agent may be administered in combination either concomitantly (e.g., as a mixture), separately, but simultaneously (e.g., via separate intravenous lines), or sequentially (e.g., one agent is administered first followed by administration of the second agent). Thus, the term combination is used to refer to concomitant, simultaneous or sequential administration of the PTPRS activating agent and a secondary active agent. In other embodiments, where the PTPRS activating agent and the secondary active agent are administered sequentially, the PTPRS activating agent is administered at a first time point and the secondary active agent is administered at a second time point, wherein the first time point precedes the second time point. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly). Thus, in some embodiments, the PTPRS activating agent and the secondary active agent are administered simultaneously or sequentially.

According to the methods provided herein, the subject is administered an effective amount of a PTPRS activating agent provided herein alone or in combination with a secondary active therapeutic agent. An “effective amount” is an amount sufficient to accomplish a stated purpose (e.g. achieve the effect for which it is administered, treat a disease (e.g., lupus), induce PTPRS declustering, induce PTPRS activity, reduce one or more symptoms of a disease or condition). An example of an “effective amount” is an amount sufficient to contribute to the treatment, prevention, or reduction of a symptom or symptoms of a disease (e.g., lupus), which could also be referred to as a “therapeutically effective amount.” A “reduction” of a symptom or symptoms (and grammatical equivalents of this phrase) means decreasing of the severity or frequency of the symptom(s), or elimination of the symptom(s). Guidance can be found in the literature for appropriate dosages for given classes of pharmaceutical products. For example, for the given parameter, a therapeutically effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

In other embodiments, the PTPRS activating agent is administered in an amount between about 0.5 mg/kg and 300 mg/kg. In yet other embodiments, the PTPRS activating agent is administered in an amount of between about 10 mg/kg and 300 mg/kg. In still other embodiments, the PTPRS activating agent is administered at an amount of about 1 mg/kg. In some embodiments, the PTPRS activating agent is administered at an amount of about 5 mg/kg. In still other embodiments, the PTPRS activating agent is administered at an amount of about 10 mg/kg. In some embodiments, the PTPRS activating agent is administered at an amount of about 20 mg/kg, about 30 mg/kg, or about 40 mg/kg. In other embodiments, the PTPRS activating agent is administered at an amount of about 50 mg/kg, about 60 mg/kg, about 70 mg/kg, about 80 mg/kg, about 90 mg/kg, about 100 mg/kg, about 200 mg/kg, or about 300 mg/kg. It is understood that where the amount is referred to as “mg/kg”, the amount is milligram per kilogram body weight of the subject being administered with the PTPRS activating agent.

In other embodiments, the PTPRS activating agent is administered at an amount of about 0.5 mg/kg, 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg or 300 mg/kg. In some embodiments, the PTPRS activating agent is administered at an amount of about 1 mg/kg. In some embodiments, the PTPRS activating agent is administered at an amount of about 1 mg/kg to 2 mg/kg. In other embodiments, the PTPRS-proteoglycan inhibitory agent is administered at an amount of about 1 mg/kg to 3 mg/kg, about 1 mg/kg to 4 mg/kg or about 1 mg/kg to 5 mg/kg.

In certain embodiments, the PTPRS activating agent is administered at an amount of about 10 mg BID, 20 mg BID, 30 mg BID, 40 mg BID, 50 mg BID, 60 mg BID, 70 mg BID, 80 mg BID, 90 mg BID, 100 mg BID, 110 mg BID, 120 mg BID, 130 mg BID, 140 mg BID, 150 mg BID, 160 mg BID, 170 mg BID, 180 mg BID, 190 mg BID, 200 mg BID, 210 mg BID, 220 mg BID, 230 mg BID, 240 mg BID, 250 mg BID, 260 mg BID, 270 mg BID, 280 mg BID, 290 mg BID, or 300 mg BID. In embodiments, the PTPRS activating agent is administered at an amount of about 10 mg BID. In one embodiment, the PTPRS activating agent is administered at an amount of about 20 mg BID. In another embodiment, the PTPRS activating agent is administered at an amount of about 30 mg BID. In another embodiment, the PTPRS-proteoglycan inhibitory agent is administered at an amount of about 40 mg BID. In another embodiment, the PTPRS activating agent is administered at an amount of about 50 mg BID. In another embodiment, the PTPRS activating agent is administered at an amount of about 60 mg BID. In another embodiment, the PTPRS activating agent is administered at an amount of about 70 mg BID. In another embodiment, the PTPRS-proteoglycan inhibitory agent is administered at an amount of about 80 mg BID. In another embodiment, the PTPRS activating agent is administered at an amount of about 90 mg BID. In another embodiment, the PTPRS activating agent is administered at an amount of about 100 mg BID. It is understood that where the amount is referred to as “BID” which stands for “bis in die”, the amount is administered twice a day.

In some embodiments, the PTPRS activating agent is administered at an amount of about 110 mg BID. In another embodiment, the PTPRS activating agent is administered at an amount of about 120 mg BID. In another embodiment, the PTPRS activating agent is administered at an amount of about 130 mg BID. In another embodiment, the PTPRS-proteoglycan inhibitory agent is administered at an amount of about 140 mg BID. In another embodiment, the PTPRS activating agent is administered at an amount of about 150 mg BID. In another embodiment, the PTPRS activating agent is administered at an amount of about 160 mg BID. In another embodiment, the PTPRS activating agent is administered at an amount of about 170 mg BID. In another embodiment, the PTPRS-proteoglycan inhibitory agent is administered at an amount of about 180 mg BID. In another embodiment, the PTPRS activating agent is administered at an amount of about 190 mg BID. In another embodiment, the PTPRS activating agent is administered at an amount of about 200 mg BID. In another embodiment, the PTPRS activating agent is administered at an amount of about 210 mg BID. In another embodiment, the PTPRS activating agent is administered at an amount of about 220 mg BID. In another embodiment, the PTPRS activating agent is administered at an amount of about 230 mg BID. In another embodiment, the PTPRS-proteoglycan inhibitory agent is administered at an amount of about 240 mg BID. In another embodiment, the PTPRS activating agent is administered at an amount of about 250 mg BID. In another embodiment, the PTPRS activating agent is administered at an amount of about 260 mg BID. In another embodiment, the PTPRS activating agent is administered at an amount of about 270 mg BID. In another embodiment, the PTPRS-proteoglycan inhibitory agent is administered at an amount of about 280 mg BID. In another embodiment, the PTPRS activating agent is administered at an amount of about 290 mg BID. In another embodiment, the PTPRS activating agent is administered at an amount of about 300 mg BID. It is understood that where the amount is referred to as “BID” which stands for “bis in die”, the amount is administered twice a day.

In yet other embodiments, the PTPRS activating agent is administered at an amount of about 10 mg QD, 20 mg QD, 30 mg QD, 40 mg QD, 50 mg QD, 60 mg QD, 70 mg QD, 80 mg QD, 90 mg QD, 100 mg QD, 110 mg QD, 120 mg QD, 130 mg QD, 140 mg QD, 150 mg QD, 160 mg QD, 170 mg QD, 180 mg QD, 190 mg QD, 200 mg QD, 210 mg QD, 220 mg QD, 230 mg QD, 240 mg QD, 250 mg QD, 260 mg QD, 270 mg QD, 280 mg QD, 290 mg QD, or 300 mg QD. In another embodiment, the PTPRS activating agent is administered at an amount of about 10 mg QD. In another embodiment, the PTPRS activating agent is administered at an amount of about 20 mg QD. In another embodiment, the PTPRS activating agent is administered at an amount of about 30 mg QD. In another embodiment, the PTPRS activating agent is administered at an amount of about 40 mg QD. In another embodiment, the PTPRS activating agent is administered at an amount of about 50 mg QD. In another embodiment, the PTPRS activating agent is administered at an amount of about 60 mg QD. In another embodiment, the PTPRS-proteoglycan inhibitory agent is administered at an amount of about 70 mg QD. In another embodiment, the PTPRS activating agent is administered at an amount of about 80 mg QD. In another embodiment, the PTPRS activating agent is administered at an amount of about 90 mg QD. In another embodiment, the PTPRS activating agent is administered at an amount of about 100 mg QD. In another embodiment, the PTPRS activating agent is administered at an amount of about 110 mg QD. In another embodiment, the PTPRS activating agent is administered at an amount of about 120 mg QD. In another embodiment, the PTPRS activating agent is administered at an amount of about 130 mg QD. In another embodiment, the PTPRS-proteoglycan inhibitory agent is administered at an amount of about 140 mg QD. In another embodiment, the PTPRS activating agent is administered at an amount of about 150 mg QD. In another embodiment, the PTPRS activating agent is administered at an amount of about 160 mg QD. In another embodiment, the PTPRS activating agent is administered at an amount of about 170 mg QD. In another embodiment, the PTPRS-proteoglycan inhibitory agent is administered at an amount of about 180 mg QD. In another embodiment, the PTPRS activating agent is administered at an amount of about 190 mg QD. In another embodiment, the PTPRS activating agent is administered at an amount of about 200 mg QD. In another embodiment, the PTPRS activating agent is administered at an amount of about 210 mg QD. In another embodiment, the PTPRS activating agent is administered at an amount of about 220 mg QD. In another embodiment, the PTPRS activating agent is administered at an amount of about 230 mg QD. In another embodiment, the PTPRS-proteoglycan inhibitory agent is administered at an amount of about 240 mg QD. In another embodiment, the PTPRS activating agent is administered at an amount of about 250 mg QD. In another embodiment, the PTPRS activating agent is administered at an amount of about 260 mg QD. In another embodiment, the PTPRS activating agent is administered at an amount of about 270 mg QD. In another embodiment, the PTPRS activating agent is administered at an amount of about 280 mg QD. In another embodiment, the PTPRS activating agent is administered at an amount of about 290 mg QD. In another embodiment, the PTPRS activating agent is administered at an amount of about 300 mg QD. It is understood that where the amount is referred to as “QD” which stands for “quaque die”, the amount is administered once a day.

In some embodiments, the PTPRS activating agent includes an amino acid sequence set forth in SEQ ID NO:1. In some embodiments, the PTPRS activating agent includes an amino acid sequence set forth as SEQ ID NO: 2. In some embodiments, the amino acid sequence of the PTPRS activating agent can have about 60%, 65%, 70%, 75%, 80%, 85%, 90%, 91%, 92%, 93%, 94%, 95%, 96%, 97%, 988, 99%, or higher sequence identity to SEQ ID NO:1 or SEQ ID NO: 2.

One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, isoforms and alleles disclosed herein. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Other pharmaceutically acceptable carriers known to those of skill in the art are suitable for compositions of the present application.

The compositions for administration will commonly comprise an agent as described herein dissolved in a pharmaceutically acceptable carrier, typically an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the subject's needs.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component.

Thus, the compositions can be administered in a variety of unit dosage forms depending upon the method of administration. For example, unit dosage forms suitable for oral administration include, but are not limited to, powder, tablets, pills, capsules and lozenges.

Compositions can be formulated to provide quick, sustained or delayed release after administration by employing procedures known in the art. Certain carriers may be more preferable depending upon, for instance, the route of administration and concentration of composition being administered. Suitable formulations for use in the provided compositions can be found in Remington: The Science and Practice of Pharmacy, 21st Edition, David B. Troy, ed., Lippicott Williams & Wilkins (2005).

Provided herein are kits comprising one or more of the provided compositions and instructions for use. Optionally, the kit comprises one or more doses of an effective amount of a composition comprising a PTPRS activating agent and anti-B cell agents, for example a TNF inhibitor or IL-6 inhibitor. Optionally, the kit comprises a non-enzymatic recombinant protein comprising an amino acid sequence of an extracellular domain of PTPRS as described herein or a subsequence, portion, homologue, isoform, variant or derivative thereof. Optionally, the kit comprises one or more portions of the extracellular domain of PTPRS. Optionally, the composition or protein is present in a container (e.g., vial or packet). Optionally, the kit comprises one or more additional agents for treating or preventing one or more symptom of an inflammatory and/or autoimmune disease. Optionally, the kit comprises a means of administering the composition, such as, for example, a syringe, needle, tubing, catheter, patch, and the like. The kit may also comprise formulations and/or materials requiring sterilization and/or dilution prior to use.

The compositions and agents as described herein are useful for both prophylactic and therapeutic treatment. For prophylactic use, a therapeutically effective amount of the agents described herein are administered to a subject prior to or during early onset (e.g., upon initial signs and symptoms of an autoimmune disease). Therapeutic treatment involves administering to a subject a therapeutically effective amount of the agents described herein after diagnosis or development of disease.

The provided polypeptides, agents and compositions are for use in the treatment of a subject who has or is at risk of developing an autoimmune disease, including for example a TLR9-associated disease or disorder or a disease with an IFN signature.

The invention is illustrated in the following examples, which are provided by way of illustration and are not intended to be limiting.

### Examples

Stimulation of mouse pDC with CpGA±Ig1&2. Splenocytes were isolated from spleens of Balb/c mice, mashed and passed through nylon filter by centrifuging with a syringe piston. Red blood cells (RBCs) were lysed by incubating the cell resuspension with RBC lysis buffer and purified by centrifugation. The remaining cells were filtered with filter-top tubes again and counted. pDC were isolated by FACS by resuspending in Sorting Buffer (1% FBS, 25 mM Hepes, 1 mM EDTA in PBS). Fc block was applied to a concentration of 1:200. The cell resuspension was stained with the following antibody panel for 30 minutes on ice away from light: MHCII eFluor 450 (1:200), PDCA1 FITC (1:200), CD11c APC (1:100), TCRb PerCPCy5.5 (1:200), CD19 PE (1:200), CD8 PECy7 (1:500), pDC was sorted as TCRb−, CD19−, PDCA1+, CD11c+.

pDCs sorted, as above, were either unstimulated or treated with 20 nM Ig1&2, 5M CpGA, or a combination of CpGA and Ig1&2.

qPCR of IFNα. RNA was extracted from the cells and reverse transcribed into cDNA. 1:2 diluted cDNA was used for qPCR with primer pairs of IFNα and GAPDH.

Injection of Balb/c mice with CpGA±Ig1&2. 2 mice were treated with 500 μg Ig1&2 and then 5 μg CpGA via R.O. injection while another 2 with treated with only CpGA. The mice were then euthanized 1 hour after the injection.

IFNalpha ELISA. Serum was harvested by incubating the blood collected before and after treatment at 37° C. for 30 minutes and centrifuged. 1:3 diluted serum was used in IFNα Platinum ELISA Kit (eBioscience, San Diego, CA).

Injection of BXSB mice with Ig1&2. 8 mice total were bought from the Jackson Laboratory, in which 4 mice were injected with vehicle and 4 with His-tagged Ig1&2 via R.O. route every other day starting from 3-month-old. The mice were euthanized at 4-month-old. Urine was collected before and after the treatment for proteinuria measurement. Spleens were harvested and weighed after euthanasia. The splenocytes were isolated for analysis using flow cytometry. Both kidneys were harvested after euthanasia. One was used for histology and the other for gene expression study using qPCR. Blood was collected after the treatment for IgG subtyping via ELISA.

The kidney used for histology was fixed in 10% zinc formalin and embedded in paraffin. The sections were stained with Periodic acid-Schiff (PAS). The histology slides were scored 1-3 according to the following criteria: minimal damage to glomerular basement membranes (GBM) and little to no cellular infiltrates, intermediate degree of cellular infiltration with moderate damage to GBM, extensive cellular infiltration and severe destruction of GBM.

The kidney used for gene expression study was fast frozen in liquid nitrogen and stored at −80° C. until use. RNA was extracted from homogenized kidney and reverse transcribed into cDNA. 1:2 diluted cDNA was used for qPCR with primer pairs of IFIT, IRF, OAS3 and GAPDH.

pDC isolation. PBMCs were isolated from whole blood using density gradient centrifugation with ficoll/histopaque. pDC was isolated from PBMC using Human pDC enrichment kit (Stemcell).

pDC stimulation. The enriched pDCs were either unstimulated or treated with 20 nM Ig1&2, 5 μM CpGA, or both respectively for 12 hours. The cell viability was measured before and after the CpG stimulation using Trypan Blue on a Vi-CELL XR 2.03.

qPCR of IFN alpha. RNA was extracted from the cells and reverse transcribed into cDNA. 1:2 diluted cDNA was used for qPCR with primer pairs of IFNα and GAPDH.

Expression construct for PTPRS Ig1&2. mRNA encoding RPTPσ Ig1&2 (amino acids 30-231 of NCBI Ref. Seq. NM_130853.2) was cloned into the pHLsec vector, introducing an N-terminal secretion signal sequence and a C-terminal hexahistadine tag.

Isolation of PTPRS SD Constructs. These methods can be used to isolate any of the PTPRS activating agents of this disclosure. The methods presented are described for Ig1&2, but are easily applicable to, for example, PTPRσ Ig1&2, PTPRσ Ig1&3, PTPRσ Ig2&3, and PTPRσ Ig1&2&3 and the other constructs utilizing Ig-like extracellular domains of PTPRS described herein.

Isolation of PTPRS Ig1&2 as a secreted protein from HEK293T cells. HEK293T cells are grown to 80-90% confluence in DMEM media containing 10% FBS. Cells are transfected with polyethylene imine: 25 μg of DNA and 37.5 μl 1 mg/ml per 150 mm plate polyethylene imine are mixed in 2.5 ml DMEM and incubated for 10-20 minutes. The media is replaced with fresh DMEM containing 2% FBS and the transfection reaction is added. After 2-3 days the supernatant is harvested and filtered. Optionally, more media is added to the cells and harvested after 2-3 days to increase protein yields.

Isolation of PTPRS Ig1&2 as a C-terminal 6×His-tag fusion. The supernatant from the above-described cells is applied to 1-1.5 ml Ni-IDA affinity beads. The beads are washed with 2 column volumes of 20 mM Tris pH 8 0.15 M NaCl and >5 column volumes of 20 mM Tris pH 8 0.5 M NaCl, 25 mM imidazole pH 8.0. The protein is eluted with 4× 1 ml 125 mM NaCl, 250 mM imidazole pH 8.0. The eluted fractions are loaded on a 1 ml heparin-sepharose HP column (Cytiva) using a syringe. The protein is eluted with a gradient from 125 mM to 1 M NaCl on a FPLC system. Proteins is purified to homogeneity as assessed by SDS-PAGE.

1. A method of treating an autoimmune disease having an interferon (IFN) signature in a subject, the method comprising administering to the subject a therapeutically effective amount of a PTPRS activating agent that inhibits the association of proteoglycans with PTPRS on plasmacytoid dendritic cells (pDCs), wherein administration treats the autoimmune disease in the subject.

2. The method of claim 1, wherein the autoimmune disease is lupus.

3. The method of claim 1, wherein the autoimmune disease is systemic lupus erythematosus.

4. (canceled)

5. The method of claim 1, wherein the subject has a TLR9-associated disease or disorder that causes increased IFNα production.

6. (canceled)

7. The method of claim 1, wherein the PTPRS activating agent comprises a soluble extracellular domain of PTPRS.

8. The method of claim 7, wherein the agent is selected from the group consisting of PTPRS Ig1&2, PTPRS Ig1&2&3, PTPRS Ig2&3, and PTPRS Ig1&3 and multimers thereof.

9. The method of claim 8, wherein the agent is PTPRS Ig1&2.

10. The method of claim 8, wherein the agent comprises multimers of Ig1&2 wherein the multimers are linked by a peptide linker.

11. A composition comprising a PTPRS activating agent.

12. The composition of claim 11, wherein the PTPRS activating agent inhibits the association of proteoglycans with PTPRS on pDCs.

13. The composition of claim 11, wherein the PTPRS activating agent comprises a soluble extracellular domain of PTPRS.

14. The composition of claim 13, wherein the agent is selected from the group consisting of PTPRS Ig1&2, PTPRS Ig1&2&3, PTPRS Ig2&3, and PTPRS Ig1&3 and multimers thereof.

15. The composition of claim 14, wherein the agent is PTPRS Ig1&2.

16. The method of claim 15, wherein the agent comprises multimers of Ig1&2 wherein the multimers are linked by a peptide linker.


SEQUENCE LISTING (14 sequences)

SEQ ID NO: 1 - protein, 97 aa, synthetic construct
EEPRFIKEPKDQIGVSGGVASFVCQATGDPKPRVTWNKKGKKVNSQRFETIEFDESAGAVLRIQPLRTPRDENVYECVAQNSVGEITVHAKLTVLRE

SEQ ID NO: 2 - protein, 113 aa, synthetic construct
DQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRVRRVA

SEQ ID NO: 3 - protein, 91 aa, synthetic construct
VRRVAPRFSILPMSHEIMPGGNVNITCVAVGSPMPYVKWMQGAEDLTPEDDMPVGRNVLELTDVKDSANYTCVAMSSLGVIEAVAQITVKS

SEQ ID NO: 4 - protein, 197 aa, synthetic construct
EEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVR

SEQ ID NO: 5 - protein, 197 aa, synthetic construct
EEPPRFIREPKDQIGVSGGVASFVCQATGDPKPRVTWNKKGKKVNSQRFETIDFDESSGAVLRIQPLRTPRDENVYECVAQNSVGEITIHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVR

SEQ ID NO: 6 - protein, 197 aa, synthetic construct; identical to SEQ ID NO: 4

SEQ ID NO: 7 - protein, 197 aa, synthetic construct; identical to SEQ ID NO: 4

SEQ ID NO: 8 - protein, 197 aa, synthetic construct
EEPPRFIKEPKDQIGVSGGVASFVCQATGDPKPRVTWNKKGKKVNSQRFETIEFDQSAGAVLRIQPLRTPRDENVYECVAQNSAGEVTVHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVR

SEQ ID NO: 9 - protein, 197 aa, synthetic construct
EEPPRFIKEPKDQIGVSGGVASFVCQATGDPKPRVTWNKKGKKVNSQRFETIEFDGSAGAVLRIQPLRTPRDENVYECVAQNSVGEITVHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVR

SEQ ID NO: 10 - protein, 197 aa, synthetic construct
EEPPRFIREPKDQIGVSGGVASFVCQATGDPKPRVTWNKKGKKVNSQRFETIDFDESSGAVLRIQPLRTPRDENVYECVAQNSVGEITVHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVR

SEQ ID NO: 11 - protein, 197 aa, synthetic construct
ESPPVFIKKPVDQIGVSGGVASFVCQATGDPKPRVTWNKKGKKVNSQRFETIEFDESAGAVLRIQPLRTPRDENIYECVAQNPHGEVTVHAKLTVLREDQLPPGFPNIDMGPQLKVVERTRTATMLCAASGNPDPEITWFKDFLPVDPSTSNGRIKQLRSGGLQIESSEETDQGKYECVASNSAGVRYSSPANLYVR

SEQ ID NO: 12 - protein, 197 aa, synthetic construct
EEPPRFIREPKDQIGVSGGVASFVCQATGDPKPRVTWNKKGKKVNSQRFETIEFDGSAGAVLRIQPLRTPRDENVYECVAQNSVGEITVHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVR

SEQ ID NO: 13 - protein, 288 aa, synthetic construct
EEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRVRRVAPRFSILPMSHEIMPGGNVNITCVAVGSPMPYVKWMQGAEDLTPEDDMPVGRNVLELTDVKDSANYTCVAMSSLGVIEAVAQITVKS

SEQ ID NO: 14 - protein, 288 aa, synthetic construct
EEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSGALQIESSEETDQGKYECVATNSAGVRYSSPANLYVRVQNVAPRFSILPMSHEIMPGGNVNITCVAVGSPMPYVKWMQGAEDLTPEDDMPVGRNVLELTDVKDSANYTCVAMSSLGVIEAVAQITVKS